(19) United States
(12) Patent Application Publication
(10) Pub. No.: US 2008/0104736 A1
(43) Pub. Date: May 8, 2008

(54) Antiseptic glove and method of use

(76) Inventor: Jeffrey P. Legaard, Marcola, OR (US)

Correspondence Address: Legaard, Jeffrey P., PO Box 1161, Marcola, OR, 97454-1161, US

(21) Appl. No.: 11/593,392

(22) Filed: Nov. 6, 2006

(51) Int. Cl. A41D 13/08; A41D 19/01; A41D 19/00

(52) U.S. Cl. 2/16; 2/158; 2/167

(57) ABSTRACT

An apparatus and method of substantial sterilization of human hands is disclosed. Hand coverings such as gloves or mitts containing an antiseptic agent may be worn for a period of time necessary to kill or disable a desired percentage of microorganisms present on hands.

### BACKGROUND

Research has shown that most bacteria are transferred from the host's bodily fluids, feces, and mucous membranes, to the host's hands, to all objects contacted by those hands, such as phones, furniture, keypads, hand rails, shopping carts, pens, credit cards, money, door knobs, flush handles, hand shakes, serving utensils, container handles, etc. The recipient typically receives those bacteria through contact with any shared surface or object. From the recipient's hands the bacteria and viruses typically become internalized by contact between the recipient's hands and his or her face which occurs on average seven times per hour, and which introduces the pathogens to his or her own mucous membranes. The recipient is now a host and distributor, passing the pathogens on to every surface or object his or her hands, mucous membranes, or bodily fluids contact.

The public restroom is the most hazardous environment we face. Most public use toilets are the violent flushing type designed to prevent blockages. When a toilet flushes, the force can spread bacteria and viruses over a fifteen foot radius, filling the air and covering all surfaces. The soap provided even if it is marketed as “antibacterial”, is not strong enough to have an antiseptic effect. Moreover, antiseptic hand washing would require a real antibacterial soap, and twenty minutes of serious scrubbing to reach all hand and finger surfaces. Even if an operating room quality wash were possible, we would still be covered by airborne mucous membrane/fecal bacteria if a toilet flushing occurred, and then have to leave the rest room area without touching any surface on the way out. In short, we go into the most polluted room, to perform an ineffective ceremony, and then have dinner.

The H5N1 Avian Flu virus is frightening example of the world-wide lack of public health planning by governments, particularly the United States. With a pathogen unknown to the human species, no vaccine, and only one possible treatment medication our only defense will be a good offense.

### SUMMARY OF THE INVENTION

A glove, mitt, or other hand covering that covers all or parts of the hand containing a means of performing substantial antisepsis of the hand.

### DETAILED DESCRIPTION OF THE PREFERRED EMBODIMENTS

An apparatus and method for killing or disabling microbial organisms present on human hands and thus performing substantial antisepsis of the hands includes a partial or whole hand covering containing a broad spectrum anti-bacterial/anti-viral, or similar antiseptic agent. The hand covering may be a fingered glove or a mitt or other such hand covering device. The glove may be designed for single use followed by disposal, or may be re-used, and may be made from a material such as a vinyl polymer. Surfaces of the interior of the glove will be substantially coated with a topical antiseptic substance that does not require the use of soap or water in order to provide a cleansing or antiseptic effect. This substance may also contain a skin moisturizer, hypo-allergenic agents, fragrances or other olfactory agents, emollients, or other substances which may aid comfort and skin tolerance. To substantially sterilize hands, hand surfaces must be in direct contact with the antiseptic agent, including the backs of the hands and between each finger. A fingered glove may provide this kind of total hand and finger coverage. A mitt or other such hand covering may also provide suitable hand coverage.

A broad spectrum alcohol based antiseptic will kill at least 99% of harmful pathogens with only fifteen to thirty seconds of contact. The glove disclosed herein may be worn for that period of time. Alcohol based sterilization products are typically manufactured as gels and foams, and include the commercial products Purell by Pfizer, Avagard D by 3M, Triad Alcohol Gel Sanitizer, Sween Isagel No Rinse Lotion Hand Gel Sanitizer, Triad Alcohol Gel Sanitizer, Chester April Guard Instant Hand Sanitizer, etc. These share alcohol as the active ingredient as ethanol, or isopropanol, at usual concentrations of 60% to 70%. They also contain humectants and emollients that moisturize the skin and counteract the drying effects of the alcohol. Some also contain perfumes. In 1994 the FDA classified ethanol alcohol at a 60% to 95% concentration, as a Category I sterilization agent.

The glove may be intended to be used once or re-used, and may have substantially all interior surfaces covered with a chemical that acts as an antiseptic. The glove enables the chemical topical antiseptic to reach substantially all surfaces of the hand and fingers, and permits a user to sterilize or substantially sterilize his or her hands without soap or water by maintaining substantial contact between hand surfaces and the antiseptic agent for the entire period of time the gloves are worn. The gloves may be worn by a user for an appropriate period of time, such as fifteen to thirty seconds, to kill or disable a desired percentage of pathogenic or other microbial organisms. This period of time may vary depending on the topical antiseptic agent employed or the organisms present.

All surfaces may be in contact with the antiseptic agent. This includes the backs of the hands and between each finger. A glove may provide this kind of total hand and finger coverage. A broad spectrum instant antiseptic can kill harmful bacteria with less than sixty seconds of contact. Consistent with the method of using these gloves, the user may keep the gloves on for fifteen or more seconds, and may optionally apply light friction or contact between his or her hands by rubbing or massaging them while wearing the gloves. Wearing the gloves will provide contact with the topical antiseptic for a period of time effective to kill or disable the desired percentage of microbial organisms present on the hands, and may sterilize or substantially sterilize the hands. The gloves may be worn for a period of time suitable to kill the desired percentage of pathogens or other microbes. The sterilization of the hands and fingers can occur through use of these sanitizing disposable gloves at the entrance to places where people congregate, including schools, hospitals, airports, restaurants, theaters, stores, offices, factories, shopping malls, etc. Users may put the gloves on at the entrance, then proceed inside, where fifteen to thirty seconds further in, receptacles for glove discard may be placed. If a public restroom is used, another pair may be worn and discarded in fifteen to thirty seconds.

These gloves can present an investment that can not only prevent a user's victimization by disease, but also prevent a user from bringing it home to his or her family. The method disclosed herein is a practice that can reduce sick leave for workers and students, make public places more acceptable, and may prevent large out breaks of disease. And just as importantly, it's a real thing that each of us can do to help ourselves and each other.

New and more deadly bacteria and virus forms are developing world wide, and the global economy is making them all local. The research, development, and production of vaccines lags behind, and takes a distant back seat to the development and marketing of designer drugs related to mood, sex, and weight loss. It is increasing apparent that individuals will need to take more responsibility for their own health and survival, that of their loved ones, and that of their fellow citizens. Magic medical bullets are going to get harder to find, and harder to afford. Sterile hands will not only be an effective disease preventative, but a real and concrete act that we can all perform, giving each of us a positive psychological sense of personal responsibility and personal control.

The preceeding terms and descriptions are intended for illustration only. The scope of this invention is intended to be limited only by the claims which follow.

I claim:

1. A hand covering containing an antiseptic agent.

2. The hand covering of claim 1 wherein said antiseptic agent is alcohol based.

3. The hand covering of claim 1 wherein said hand covering is a fingered glove.

4. The hand covering of claim 1 wherein said hand covering is a mitt.

5. A hand covering having interior surfaces wherein said interior surfaces are substantially covered in an antiseptic agent.

6. The hand covering of claim 5 wherein said antiseptic agent is alcohol based.

7. The hand covering of claim 5 wherein said hand covering is a fingered glove.

8. The hand covering of claim 5 wherein said hand covering is a mitt.

9. The hand covering of claim 5 wherein said antiseptic agent is combined with a moisturizing substance.

10. The hand covering of claim 5 wherein said antiseptic agent is combined with at least one of a group containing substances having olfactory, moisturizing, humectant, and emollient properties.

11. The hand covering of claim 5 wherein said hand covering is used once.

12. The hand covering of claim 5 wherein said hand covering is re-usable.

13. A method of substantially sterilizing human hands comprising: providing hand coverings containing an antiseptic agent, covering said hands with said hand coverings for a period of time sufficient to accomplish substantial sterilization, and removing said hand coverings.

14. The method of claim 13 wherein said hand coverings are fingered gloves.

15. The method of claim 13 wherein said hand coverings are mitts.

16. The method of claim 13 wherein said antiseptic agent is alcohol based.

17. The method of claim 13 wherein said antiseptic agent is combined with a moisturizing substance.
